(19) United States
(12) Patent Application Publication
(10) Pub. No.: US 2026/0023040 A1
(43) Pub. Date: Jan. 22, 2026

(54) DEVICE AND METHOD FOR MEASURING COUPLING RELATIONSHIP BETWEEN ICE CONTENT AND DEFORMATION OF FROZEN SOIL

(71) Applicant: SUN YAT-SEN UNIVERSITY, Guangzhou (CN)

(72) Inventors: Dingfeng Cao, Guangzhou (CN); Fuming Wang, Guangzhou (CN); Chengchao Guo, Guangzhou (CN); Fan Yang, Guangzhou (CN); Lei Qin, Guangzhou (CN)

(21) Appl. No.: 19/001,453

(22) Filed: Dec. 25, 2024

(30) Foreign Application Priority Data
Jul. 19, 2024 (CN) 202410971068.8

(51) Int. Cl. G01N 25/58; G01N 3/18; G01N 33/24

(52) U.S. Cl. CPC G01N 25/58; G01N 3/18; G01N 33/24; G01N 2203/0244

(57) ABSTRACT

Provided are a device and method for measuring a coupling relationship between an ice content and a deformation of a frozen soil. The device includes: a soil sample container; a first thermal conductive disc; an annular support provided therein with a porous plate, where an upper end surface of the porous plate, a lower end surface of the first thermal conductive disc, and an inner wall of the soil sample container cooperate to form an accommodation chamber for accommodating a target in-situ frozen soil; a second thermal conductive disc located below the porous plate, where a lower end surface of the porous plate, an inner wall of the annular support, and an upper end surface of the second thermal conductive disc cooperate to form a water storage chamber; and a loading assembly configured to apply a load to the target in-situ frozen soil inside the accommodation chamber.

### CROSS REFERENCE TO RELATED APPLICATIONS

The present application claims the benefit of Chinese Patent Application No. 202410971068.8 filed on Jul. 19, 2024, the contents of which are incorporated herein by reference in their entirety.

### TECHNICAL FIELD

The present disclosure relates to the technical field of soil monitoring, and in particular to a device and method for measuring a coupling relationship between an ice content and a deformation of a frozen soil.

### BACKGROUND

Frozen soil is formed due to long-term interactions of complex atmospheric systems. Frozen soil is highly sensitive to temperature, and seasonal changes in climate can cause frost heave and thaw settlement deformations of frozen soil, posing a serious threat to the safe operation of road engineering. The deformation of frozen soil is closely related to its ice content. Therefore, understanding the coupling relationship between the ice content and deformation of frozen soil is of great significance for theoretical and experimental research on frozen soil areas.

The ice content of frozen soil is measured by methods such as expansion method, dielectric spectroscopy method, heat pulse method, nuclear magnetic resonance (NMR) method, and distributed fiber optic measurement method. The freeze-thaw deformation of frozen soil is mainly predicted through experience and theoretical models. The solid ice content and strain in frozen soil are interdependent. However, at present, laboratory experiments can only measure the ice content or freeze-thaw deformation of frozen soil alone, and cannot measure the coupling between the ice content and deformation of frozen soil.

### SUMMARY

An objective of the present disclosure is to provide a device and method for measuring a coupling relationship between an ice content and a deformation of a frozen soil. The present disclosure can achieve synchronous measurement of the ice content and the deformation of the frozen soil, and acquire a quantitative coupling relationship between the ice content and the deformation.

To achieve the above objective, the present disclosure provides a device for measuring a coupling relationship between an ice content and a deformation of a frozen soil, including:
a soil sample container, being hollow inside and open at upper and lower ends;
a first thermal conductive disc, located inside the soil sample container and close to the upper end of the soil sample container;
an annular support, located at the lower end of the soil sample container and provided therein with a porous plate, where an upper end surface of the porous plate, a lower end surface of the first thermal conductive disc, and an inner wall of the soil sample container cooperate to form an accommodation chamber for accommodating a target in-situ frozen soil;
a second thermal conductive disc, located inside the annular support and below the porous plate, where a lower end surface of the porous plate, an inner wall of the annular support, and an upper end surface of the second thermal conductive disc cooperate to form a water storage chamber; and
a loading assembly, located above the first thermal conductive disc and configured to apply a load to the target in-situ frozen soil inside the accommodation chamber.

Furthermore, a first flange is provided at a top of the soil sample container; the annular support includes a second flange and a third flange arranged in sequence from top to bottom; first screws are uniformly distributed in a circumferential direction of the first flange; the first screws pass through the first flange and the second flange in sequence, and each include upper and lower ends that are provided with a first nut and a second nut, respectively; second screws are uniformly distributed in a circumferential direction of the second flange; and the second screws pass through the second flange and the third flange in sequence, and each include upper and lower ends that are provided with a third nut and a fourth nut, respectively.

Furthermore, the loading assembly includes a connecting seat, a pressure rod, a weight element, and a pressure plate; the connecting seat is fixed to the soil sample container; the pressure rod includes one end hinged to the connecting seat and the other end provided with the weight element; an axis of the pressure rod and an axis of the soil sample container intersect and are located in a same vertical plane; the first thermal conductive disc is fixed to the pressure plate; and the pressure plate is slidably connected to the pressure rod.

Furthermore, the loading assembly further includes a guide plate, a connecting element, a connecting shaft, a guide shaft, and a linear bearing; the guide plate is fixed above the soil sample container; a center of the guide plate coincides with the axis of the soil sample container; the linear bearing is provided at a central part of the guide plate; the pressure rod is axially provided with a sliding groove; two ends of the connecting shaft pass through the sliding groove and are fixed to the connecting element; a bottom of the connecting element is fixed to the connecting shaft; and the other end of the connecting shaft is fixed to the pressure plate.

Furthermore, an end of the pressure rod is axially provided with a mounting groove for limiting the mounting of the weight element.

Furthermore, the first thermal conductive disc and the second thermal conductive discs are structurally identical; the first thermal conductive disc includes a disc body and a coil; the disc body and the soil sample container are arranged coaxially; the disc body is provided therein with a hollow chamber, and the coil is located inside the hollow chamber; and the coil includes one end provided with a thermal conductive medium inlet and the other end provided with a thermal conductive medium outlet.

Furthermore, a side wall of the soil sample container is axially provided with mounting holes that are uniformly distributed for mounting frequency domain reflectometry (FDR) sensors.

Furthermore, a base is provided at a bottom of the annular support; and a brake caster is located below the base.

Furthermore, the device further includes a distributed sensing optical cable, a pull-out tester, and a fiber optic demodulation device; the distributed sensing optical cable includes one end fixedly connected to the porous plate and the other end connected to the fiber optic demodulation device; and the pull-out tester and the distributed sensing optical cable are clamped and fixed by a fixture.

The present disclosure further provides a method for measuring a coupling relationship between an ice content and a deformation of a frozen soil, based on the device for measuring a coupling relationship between an ice content and a deformation of a frozen soil according to any one of the above paragraphs, and including the following steps:
S1: embedding one end of the distributed sensing optical cable in the target in-situ frozen soil, and fixing the one end to the porous plate; and extending the other end of the distributed sensing optical cable outside the soil sample container, and fixing the other end to an external bracket, where an axis of the distributed sensing optical cable is parallel to the axis of the soil sample container;
S2: connecting the distributed sensing optical cable to the fiber optic demodulation device; and reading, by the fiber optic demodulation device, temperature information of the distributed sensing optical cable in a length direction, thereby acquiring a temperature distribution and variation of the target in-situ frozen soil in a depth direction;
S3: arranging multiple FDR sensors in an axial direction on the side wall of the soil sample container, and comparing temperature values of the target in-situ frozen soil measured by the multiple FDR sensors with temperature values of the target in-situ frozen soil measured by the distributed sensing optical cable at a same depth;
S4: pulling, by the pull-out tester, the distributed sensing optical cable at a constant rate in different freezing stages of the target in-situ frozen soil, and recording a pulling force and displacement; and monitoring, by the fiber optic demodulation device, an axial strain distribution of the distributed sensing optical cable in the length direction in real time during pulling;
S5: adjusting, by the first thermal conductive disc and the second thermal conductive disc, a temperature of the target in-situ frozen soil;
S6: adjusting, by the loading assembly, a strain of the target in-situ frozen soil in the depth direction; and
S7: measuring a temperature, a water content, and an ice content of the target in-situ frozen soil, as well as a strain distribution and a variation of the target in-situ frozen soil in the depth direction, thereby acquiring a quantitative coupling relationship between the ice content and the deformation.

Embodiments of the present disclosure provide a device and method for measuring a coupling relationship between an ice content and a deformation of a frozen soil. Compared to the prior art, the present disclosure has the following beneficial effects. The target in-situ frozen soil is located inside the accommodation chamber. The temperature of the target in-situ frozen soil is controlled through the first thermal conductive disc and the second thermal conductive discs to simulate the external temperature environment in which the in-situ frozen soil is located. The water storage chamber is provided to replenish water into the target in-situ frozen soil. The loading assembly is provided to adjust the pressure inside the target in-situ frozen soil. By simulating the temperature environment, humidity environment, and pressure environment of the target in-situ frozen soil, the present disclosure achieves synchronous measurement of the ice content and the deformation of the frozen soil, and acquires the quantitative coupling relationship between the ice content and the deformation.

### BRIEF DESCRIPTION OF THE DRAWINGS

FIG. 1 is a structural diagram of a device for measuring a coupling relationship between an ice content and a deformation of a frozen soil according to an embodiment of the present disclosure;

FIG. 2 is an enlarged view of A shown in FIG. 1;

FIG. 3 is a sectional view of the device for measuring a coupling relationship between an ice content and a deformation of a frozen soil according to an embodiment of the present disclosure;

FIG. 4 is a structural diagram of a first thermal conductive disc of the device for measuring a coupling relationship between an ice content and a deformation of a frozen soil according to an embodiment of the present disclosure;

FIG. 5 is a partial structural diagram of the device for measuring a coupling relationship between an ice content and a deformation of a frozen soil according to an embodiment of the present disclosure; and

FIG. 6 is a structural diagram of a soil sample container of the device for measuring a coupling relationship between an ice content and a deformation of a frozen soil according to an embodiment of the present disclosure.

Reference Numerals: 1. soil sample container; 11. first flange; 100. accommodation chamber; 101. mounting hole; 2. first thermal conductive disc; 21. disc body; 22. coil; 221. thermal conductive medium inlet; 222. thermal conductive medium outlet; 3. annular support; 30. porous plate; 31. second flange; 300. water storage chamber; 32. third flange; 33. first screw; 331. first nut; 332. second nut; 34. second screw; 341. third nut; 342. fourth nut; 4. second thermal conductive disc; 5. loading assembly; 51. connecting seat; 52. pressure rod; 521. sliding groove; 522. mounting groove. 53. weight element, 54. pressure plate; 55. guide plate; 56. connecting element; 57. connecting shaft; 58. guide shaft; 59. linear bearing; 6. frequency domain reflectometry (FDR) sensor; 7. base; 8. brake caster; 301. distributed sensing optical cable; 302. fiber optic demodulation device; and 303. pull-out tester.

### DETAILED DESCRIPTION

The specific implementations of the present disclosure are described in more detail below with reference to the drawings and embodiments. The following embodiments are intended to illustrate the present disclosure, but not to limit the scope of the present disclosure.

In the description of the present disclosure, it should be understood that orientation or position relationships indicated by terms such as “upper”, “lower”, “front”, “rear”, “inside”, and “outside” are orientation or position relationships as shown in the drawings. These terms are merely intended to facilitate and simplify the description of the present disclosure, rather than to indicate or imply that the mentioned device or components must have a specific orientation or must be constructed and operated in a specific orientation. Therefore, these terms should not be understood as a limitation to the present disclosure.

It should be understood that in the description of the present disclosure, the terms such as “first” and “second” are used in the present invention to describe various information, but the information should not be limited to these terms, and these terms are only used to distinguish the same type of information from each other. For example, without departing from the scope of the present disclosure, “first” information may be referred to as “second” information, and similarly, “second” information may also be referred to as “first” information.

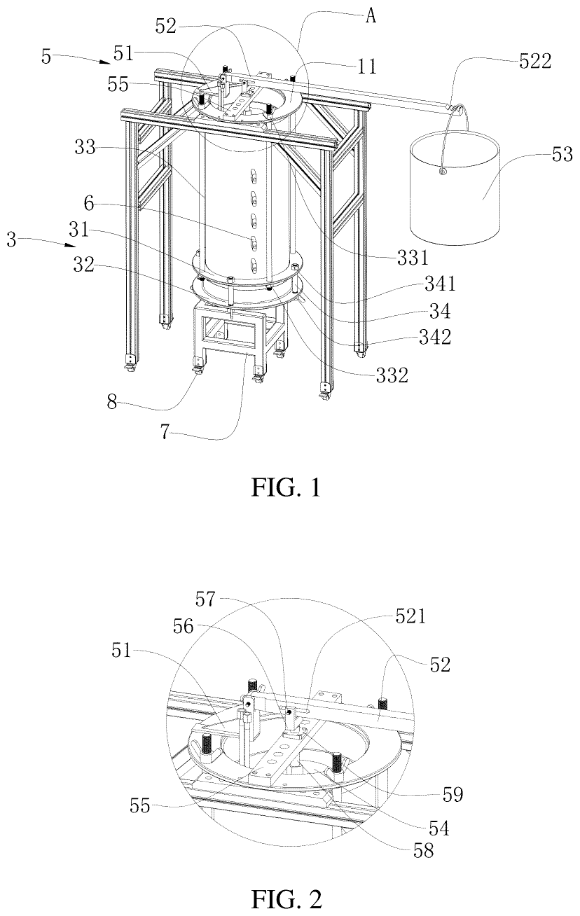

As shown in FIGS. 1 to 4, a preferred embodiment of the present disclosure provides a device for measuring a coupling relationship between an ice content and a deformation of a frozen soil, which can simulate the actual external environment of the frozen soil and improve measurement accuracy. The device includes: a soil sample container 1, a first thermal conductive disc 2, an annular support 3, a second thermal conductive disc 4, and a loading assembly 5. The soil sample container 1 is configured to accommodate a target in-situ frozen soil. The soil sample container 1 is hollow inside and open at upper and lower ends. The first thermal conductive disc 2 is located inside the soil sample container 1 and close to the upper end of the soil sample container 1. The annular support 3 is located at the lower end of the soil sample container 1. The annular support 3 is provided therein with a porous plate. In this embodiment, in order to facilitate the accommodation of the target in-situ frozen soil, an upper end surface of the porous plate, a lower end surface of the first thermal conductive disc 2, and an inner wall of the soil sample container 1 cooperate to form an accommodation chamber 100 for accommodating the target in-situ frozen soil.

The first thermal conductive disc 2 and the second thermal conductive disc 4 are configured to adjust a temperature inside the accommodation chamber 100. Specifically, the second thermal conductive disc 4 is located inside the annular support 3 and below the porous plate. In order to facilitate the replenishment of water into the target in-situ frozen soil inside the accommodation chamber 100, a lower end surface of the porous plate, an inner wall of the annular support 3, and an upper end surface of the second thermal conductive disc 4 cooperate to form a water storage chamber 300. In order to simulate the external environment pressure of the target in-situ frozen soil, the loading assembly 5 is located above the first thermal conductive disc 2 and configured to apply a load to the target in-situ frozen soil inside the accommodation chamber 100.

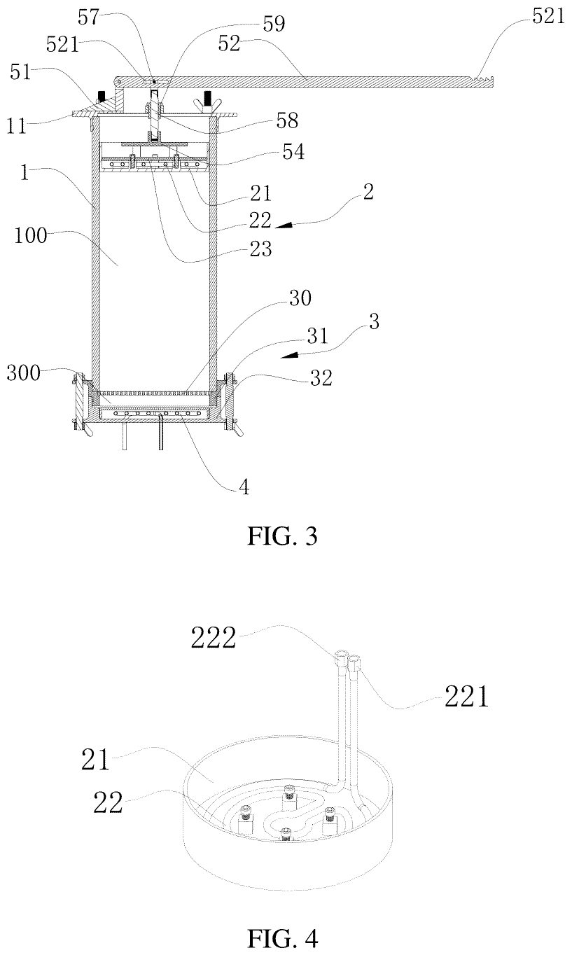

Specifically, in this embodiment, in order to facilitate the disassembly and assembly of the soil sample container 1 and the annular support 3, as shown in FIGS. 1 and 3, a first flange 11 is provided at a top of the soil sample container 1. The annular support 3 includes a second flange 31 and a third flange 32 arranged in sequence from top to bottom. First screws 33 are uniformly distributed in a circumferential direction of the first flange 11. The first screws 33 pass through the first flange 11 and the second flange 31 in sequence, and each include upper and lower ends that are provided with a first nut 331 and a second nut 332, respectively. As shown in FIG. 1, second screws 34 are uniformly distributed in a circumferential direction of the second flange 31. The second screws 34 pass through the second flange 31 and the third flange 32 in sequence, and each include upper and lower ends that are provided with a third nut 341 and a fourth nut 342, respectively. Specifically, in order to facilitate mounting limiting between the second flange 31 and the soil sample container 1, an inner wall of the second flange 31 is provided with a limiting step that achieves limiting with a bottom of the soil sample container 1. Similarly, in order to facilitate mounting limiting between the second flange 31 and the third flange 32, an inner wall of the third flange 32 is provided with a limiting step that achieves mounting limiting with the second flange 31. The second thermal conductive disc 4 is located inside the second flange plate 31. In order to seal the water storage chamber 300, an outer wall of the second flange 31 and an outer wall of the soil sample container 1 each are provided with a sealing groove for mounting a sealing ring.

Furthermore, in this embodiment, in order to facilitate the simulation of the pressure environment of the target in-situ frozen soil, the loading assembly 5 includes a connecting seat 51, a pressure rod 52, a weight element 53, and a pressure plate 54. As shown in FIGS. 2 and 3, the connecting seat 51 is fixed to the soil sample container 1. One end of the pressure rod 52 is hinged to the connecting seat 51, and the other end of the pressure rod 52 is provided with the weight element 53. An axis of the pressure rod 52 and an axis of the soil sample container 1 intersect and are located in a same vertical plane. The first thermal conductive disc 2 is fixed to the pressure plate 54. The pressure plate 54 is slidably connected to the pressure rod 52. That is, weight of the weight element 53 is adjusted such that the pressure rod 52 rotates. The pressure plate 54 slides relative to the pressure rod 52 and presses down the first thermal conductive disc 2, thereby applying a pressure to the target in-situ frozen soil inside the accommodation chamber 100. Furthermore, in order to facilitate observation of the target in-situ frozen soil inside the accommodation chamber 100, the soil sample container is made of a transparent material. Preferably, in this embodiment, the soil sample container is made of a transparent acrylic material

Furthermore, in this embodiment, in order to improve the stability of the loading assembly 5 and guide the downward pressing of the pressure plate 54, the loading assembly 5 further includes a guide plate 55, a connecting element 56, a connecting shaft 57, a guide shaft 58, and a linear bearing 59. As shown in FIGS. 1, 2, and 3, the guide plate 55 is fixed above the soil sample container 1. A center of the guide plate 55 coincides with the axis of the soil sample container 1. The linear bearing 59 is provided at a central part of the guide plate 55. The pressure rod 52 is axially provided with a sliding groove 521. Two ends of the connecting shaft 57 pass through the sliding groove 521 and are fixed to the connecting element 56. A bottom of the connecting element 56 is fixed to the connecting shaft 57. The other end of the connecting shaft 57 is fixed to the pressure plate 54. That is, after the weight of the weight element 53 is adjusted, the connecting shaft 57 slides in the sliding groove 521. The pressure rod 52 rotates upward or downward, and the guide shaft 58 slides downward or upward in the linear bearing 59, thereby pushing the pressure plate 54 to slide downward or upward.

Furthermore, in this embodiment, the weight element 53 can directly be a weight. In some embodiments, for the convenience of drawing on local resources, the weight element 53 can be replaced with a bucket filled with liquid, thereby reducing costs. Furthermore, in order to facilitate adjustment of the mounting position of the weight element 53 on the pressure rod 52, an end of the pressure rod 52 is axially provided with a mounting groove 522 for limiting the mounting of the weight element 53.

Furthermore, in order to facilitate the structural design of the first thermal conductive disc 2 and the second thermal conductive discs 4, in this embodiment, the structures of the first thermal conductive disc 2 and the second thermal conductive discs 4 are structurally identical. Specifically, as shown in FIG. 4, the first thermal conductive disc 2 includes a disc body 21 and a coil 22. The disc body 21 and the soil sample container 1 are arranged coaxially. The disc body 21 is provided therein with a hollow chamber, and the coil 22 is located inside the hollow chamber. The coil 22 includes one end provided with a thermal conductive medium inlet 221 and the other end provided with a thermal conductive medium outlet 222. In this embodiment, the thermal conductive medium inlet 221 and the thermal conductive medium outlet 222 of the first thermal conductive disc 2 are arranged vertically upward, while a thermal conductive medium inlet 221 and a thermal conductive medium outlet 222 of the second thermal conductive disc 4 are arranged vertically downward and connected to an external thermal conductive medium circulation temperature control device. Furthermore, in this embodiment, in order to facilitate the connection between the first thermal conductive disc 2 and the pressure plate 54, a cover plate 23 is provided above the disc body 21. The cover plate 23 and the pressure plate 54 are fixed by a screw.

Furthermore, in this embodiment, as shown in FIG. 6, a side wall of the soil sample container 1 is axially provided with mounting holes 101 that are uniformly distributed for mounting frequency domain reflectometry (FDR) sensors 6. The FDR sensors 6 are configured to compare temperature values of the target in-situ frozen soil measured by the multiple FDR sensors and temperature values of the target in-situ frozen soil measured by a distributed sensing optical cable at a same depth.

Furthermore, in this embodiment, in order to facilitate overall movement of the device, a base 7 is provided at a bottom of the annular support 3, and a brake caster 8 is located below the base 7.

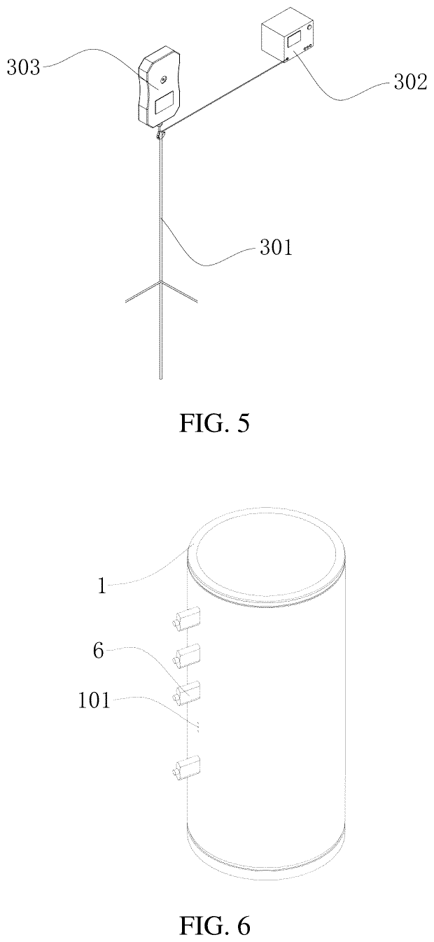

Furthermore, in this embodiment, in order to measure and analyze the ice content and deformation parameters of the frozen soil, as shown in FIG. 5, the device further includes a distributed sensing optical cable 301, a pull-out tester 303, and a fiber optic demodulation device 302. The distributed sensing optical cable 301 includes one end fixedly connected to the porous plate 30 and the other end connected to the fiber optic demodulation device 302. The pull-out tester 303 and the distributed sensing optical cable 301 are clamped and fixed by a fixture. In a possible embodiment, the device may also include coupling plates. The coupling plates are threaded on the distributed sensing optical cable 301.

The present disclosure further provides a method for measuring a coupling relationship between an ice content and a deformation of a frozen soil, based on the device for measuring a coupling relationship between an ice content and a deformation of a frozen soil according to any one of the above paragraphs, and including the following steps.

S1. One end of the distributed sensing optical cable is embedded in the target in-situ frozen soil and fixed to the porous plate. The other end of the distributed sensing optical cable extends outside the soil sample container and is fixed to an external bracket. An axis of the distributed sensing optical cable is parallel to the axis of the soil sample container.

S2. The distributed sensing optical cable is connected to the fiber optic demodulation device. The fiber optic demodulation device reads temperature information of the distributed sensing optical cable in a length direction, thereby acquiring a temperature distribution and variation of the target in-situ frozen soil in a depth direction.

S3. Multiple FDR sensors are arranged in an axial direction on the side wall of the soil sample container, and the temperature values of the target in-situ frozen soil measured by the multiple FDR sensors are compared with the temperature values of the target in-situ frozen soil measured by the distributed sensing optical cable at the same depth.

S4. The distributed sensing optical cable is pulled by the pull-out tester at a constant rate in different freezing stages of the target in-situ frozen soil, and a pulling force and displacement are recorded. An axial strain distribution of the distributed sensing optical cable in the length direction is monitored by the fiber optic demodulation device in real time during pulling.

S5. A temperature of the target in-situ frozen soil is adjusted by the first thermal conductive disc and the second thermal conductive disc.

S6. A strain of the target in-situ frozen soil in the depth direction is adjusted by the loading assembly.

S7. A temperature, a water content, and an ice content of the target in-situ frozen soil as well as a strain distribution and a variation of the target in-situ frozen soil in the depth direction are measured, thereby acquiring a quantitative coupling relationship between the ice content and the deformation.

Specifically, in the step S5, the first thermal conductive disc 2 and the second thermal conductive disc 4 achieve temperature control by an external low-temperature and constant-temperature water bath. Furthermore, in order to avoid the influence of the external environment of the device on the temperature inside the accommodation chamber, the first thermal conductive disc 2 and the second thermal conductive disc 4 are externally covered by 20 mm thick self-adhesive thermal insulation cotton. Meanwhile, a gap between the first temperature disc 2 and the soil sample container 1 is filled with thermal insulation mortar foam.

Furthermore, in the step S6, based on a principle of leverage, the weight is suspended at the end of the pressure rod 52 to simulate the pressure environment of the target in-situ frozen soil inside the accommodation chamber.

In the step S7, the acquired quantitative coupling relationship between the ice content and the deformation specifically refers to a quantitative relationship between related parameters such as water, heat, and strain of the ice content and the deformation of the frozen soil. The specific analysis and calculation are as follows.

During a freezing process, water in an unsaturated frozen soil exists in the form of liquid water and ice. According to the law of conservation of mass and considering the influence of stress and strain on the water content and ice content, the migration of water in the unsaturated frozen soil is expressed as follows:

$$\frac{\partial }{\partial t} ( {ρ}_{l} {θ}_{l} ) + \frac{\partial }{\partial t} ( {ρ}_{i} {θ}_{i} ) - \frac{\partial ε}{\partial t} ( \frac{{ρ}_{l} {θ}_{l}}{n} + \frac{{ρ}_{i} {θ}_{i}}{n} ) = - ∇ \cdot {q}_{l} \qquad ( 1 )$$

In Eq. (1), ρ₁ and ρᵢ denote densities (kg·m⁻³) of the liquid water and the ice, respectively; θ₁ and θᵢ denote volume contents (m³·m⁻³) of the liquid water and the ice, respectively; t denotes time(s); q₁ denotes a flux (kg·m⁻²·s⁻¹) of the liquid water; n denotes porosity (1); and ⊖ denotes the strain (dimensionless).

Specifically,

$${q}_{l} = {q}_{lh} + {q}_{lT} = - {K}_{lh} ( \frac{\partial h}{\partial y} + 1 ) - {K}_{lT} \frac{\partial T}{\partial y} . \qquad ( 2 )$$

In Eq. (2), K₁ₕ (m·s⁻¹) and K_{1T} (m2·K⁻¹ ·s⁻¹) denote isothermal and non-isothermal conductivity coefficients of the liquid water, respectively; y denotes a vertical coordinate of space coordinates the soil; h denotes a matric suction head (m); and T denotes temperature (K).

$${K}_{l h} = {K}_{s} {{Θ}^{l} [ 1 - {( 1 - {Θ}^{\frac{1}{m}} )}^{m} ]}^{2} \qquad ( 3 )$$

$${K}_{lT} = {K}_{l h} ( h {G}_{w T} \frac{1}{{γ}_{0}} \frac{d γ}{d T} ) \qquad ( 4 )$$

In Eq. (3), Kₛ denotes a saturated hydraulic conductivity coefficient (m·s⁻¹); Θ denotes an effective saturation (dimensionless), Θ=[1+(−αh)ⁿ]^{-m}; ^{α}(m⁻¹), n (dimensionless), m(=1-1/n, dimensionless), and l are empirical parameters, and l generally takes a value of 0.5; in Eq. (4), γ denotes a temperature dependent surface tension (kg·s⁻²); G_{wT} denotes a gravity factor (dimensionless); and γ₀ denotes a surface tensor at 25° C. (71.89 g/s²). Specifically:

$$γ = 75.6 - 0.1425 ( T - 273.15 ) - 2.38 \times {10}^{- 4} {( T - 273.15 )}^{2} \qquad ( 5 )$$

Heat is mainly transmitted through thermal conduction and convection in the unsaturated frozen soil. The energy transfer of thermal conduction is contributed by soil particles, the liquid water, and the ice, while thermal convection is controlled by the liquid water. In addition, energy generated by the strain in the unsaturated frozen soil should also be considered. Therefore, the energy conservation equation can be written in the following form:

$${C}_{e} \frac{\partial T}{\partial t} - {L}_{f} {ρ}_{i} \frac{\partial {θ}_{i}}{\partial t} = \frac{\partial }{\partial y} ( {λ}_{e} \frac{\partial T}{\partial y} ) - {C}_{l} \frac{\partial {q}_{l} T}{\partial y} + \frac{\partial {Q}_{ε}}{\partial t} \qquad ( 6 )$$

In Eq. (6), Cₑ and C₁ denote volumetric heat capacities of a medium and the liquid water, respectively (J·m⁻³·K⁻¹); L_{f} denotes latent heat of solidification of water (J·kg⁻¹); λₑ denotes a thermal conductivity coefficient of the medium (W·m⁻¹·K⁻¹); and Q_{⊖} denotes energy generated by soil deformation, specifically:

$${Q}_{ε} = ( 1 - n ) ( 2 G + 3 λ ) β ( T - {T}_{0} ) ε \qquad ( 7 )$$

where, in Eq. (7), G and λ are Lame constants (1); β denotes a coefficient of thermal expansion (1·K⁻¹); and T₀ denotes a reference temperature of β.

The heat capacity and thermal conductivity coefficient of the unsaturated frozen soil are expressed by the volume fraction of each component:

$${C}_{e} = {C}_{n} {θ}_{n} + {C}_{l} {θ}_{l} + {C}_{i} {θ}_{i} \qquad ( 8 )$$

$${λ}_{e} = {λ}_{n}^{{θ}_{n}} {λ}_{l}^{{θ}_{l}} {λ}_{i}^{{θ}_{i}} \qquad ( 9 )$$

where, in Eqs. (8) and (9), Cₙ, G₁, and Cᵢ denote volumetric heat capacities (J·m⁻³·K⁻¹) of the soil particles, water, and ice, respectively; λₙ, λ₁, and λᵢ denote thermal conductivity coefficients (W·m⁻¹·K⁻¹) of the soil particles, water, and ice, respectively; and θₙ denotes a volume content of a solid skeleton.

A relationship between an unfrozen water content and temperature in the frozen soil is as follows:

$${θ}_{l} = {( {θ}_{s} - {θ}_{r} ) [ 1 + {( - α \frac{{L}_{f} T}{g {T}_{0}} )}^{n} ]}^{- m} + {θ}_{r} \qquad ( 10 )$$

In Eq. (10), θₛ and θᵣ and or denote a saturated water content and a residual water content (m³·m⁻³), respectively.

Assuming that the soil is isotropic, an equation of static equilibrium is expressed as:

$$\frac{\partial σ}{\partial y} + ( {ρ}_{n} + {θ}_{l} {ρ}_{l} + {θ}_{i} {ρ}_{i} ) g = 0 \qquad ( 11 )$$

where, in Eq. (11), σ denotes a total stress; ρₙ denotes a density of the soil skeleton (kg·m⁻³); and g denotes a gravitational acceleration constant (m·s⁻²).

According to an elastic stress-strain relationship, a compression coefficient of a saturated soil is:

$$- \frac{d e}{d {σ}^{′}} = \frac{1 + {e}_{0}}{{E}_{S}} \qquad ( 12 )$$

In Eq. (12), σ′ denotes an effective stress; and E_{S} denotes a compression modulus of the saturated soil. For the unsaturated soil, the influence of saturation on soil strength should be considered, and a coefficient

$$χ = \frac{S}{0.4 S + 0.6}$$

is introduced to correct the compression modulus. The above equation can be simplified as:

$$d {σ}^{′} = - χ {E}_{S} \frac{d e}{1 + {e}_{0}} = - χ {E}_{S} {d}_{E} \qquad ( 13 )$$

According to Terzaghi's principle of effective stress, the total stress is a sum of the effective stress and a pore pressure:

$$σ = {σ}^{′} + [ χ {P}_{l} + ( 1 - χ ) {P}_{a} ] \qquad ( 14 )$$

where, P₁ denotes the pore water pressure (Pa); and Pₐ denotes an atmospheric pressure (Pa). Substituting Eqs. (13) and (14) into Eq. (11) yields:

$$- χ {E}_{S} \frac{d e}{1 + {e}_{0}} + ∇ \{ [ χ {P}_{l} + ( 1 - χ ) {P}_{a} ] \} = - ( {ρ}_{n} + {θ}_{l} {p}_{l} + {θ}_{i} {ρ}_{i} ) g \qquad ( 15 )$$

The calculation of the strain includes two parts: a strain caused by soil consolidation due to a decrease in the liquid water content in a thawing zone and a strain caused by an in-situ frost heave in a freezing zone.

The calculation of the strain in the thawing zone adopts a traditional soil mechanics method:

$$ε = \frac{e - {e}_{0}}{1 + {e}_{0}} \qquad ( 16 )$$

where, in Eq. (16), e denotes a void ratio after freezing (m³·m⁻³); and e₀ denotes an initial void ratio before freezing (m³·m⁻³).

The strain in the freezing zone is calculated as follows:

$$ε = \frac{{ρ}_{dry}}{2 {ρ}_{l}} ( {θ}_{t} - {W}_{k p} ) \qquad ( 17 )$$

In Eq. (17), ρ_{dry} denotes a dry density of the soil; Wₖₚ denotes an initial volumetric water content (m³·m⁻³) of the frost heave; and θₜ denotes a total volume content of water (m³·m⁻³).

There is an area of low permeability, low water content, and no frost heave, called a freezing edge, between an ice lens and a freezing front. Assuming the temperature of the freezing edge close to the ice lens is T_{f} (K), then the strain is calculated as follows:

$$ε = \{ \begin{matrix}\frac{{ρ}_{dry}}{2 {ρ}_{l}} ( {θ}_{t} - {W}_{kp} ) , T \leq {T}_{f} \\ \frac{e - {e}_{0}}{1 + {e}_{0}} , T \geq {T}_{0} \\ 0 , {T}_{f} < T < {T}_{0}\end{matrix} \qquad ( 18 )$$

According to Eqs. (1), (6), (15), and (18), the direct coupling relationship between the ice content and the deformation of the frozen soil is acquired. Meanwhile, based on real-time monitoring of the distributed sensing optical cable and the pull-out tester, the direct coupling data of the ice content and the deformation of the frozen soil is acquired.

In summary, the embodiments of the present disclosure provide a device and method for measuring a coupling relationship between an ice content and a deformation of a frozen soil. The target in-situ frozen soil is located inside the accommodation chamber 100. The temperature of the target in-situ frozen soil is controlled through the first thermal conductive disc 2 and the second thermal conductive discs 4 to simulate the external temperature environment in which the in-situ frozen soil is located. The water storage chamber 300 is provided to replenish water into the target in-situ frozen soil. The loading assembly 5 is provided to adjust the pressure inside the target in-situ frozen soil. By simulating the temperature environment, humidity environment, and pressure environment of the target in-situ frozen soil, the present disclosure achieves synchronous measurement of the ice content and the deformation of the frozen soil, and acquires the quantitative coupling relationship between the ice content and the deformation.

The above are merely descriptions of the preferred embodiments of the present disclosure. It should be noted that several improvements and replacements can be made by a person of ordinary skill in the art without departing from the technical principle of the present disclosure, and these improvements and replacements shall also be deemed as falling within the protection scope of the present disclosure.

1. A device for measuring a coupling relationship between an ice content and a deformation of a frozen soil, comprising:
a soil sample container, being hollow inside and open at upper and lower ends;
a first thermal conductive disc, located inside the soil sample container and close to the upper end of the soil sample container;
an annular support, located at the lower end of the soil sample container and provided therein with a porous plate, wherein an upper end surface of the porous plate, a lower end surface of the first thermal conductive disc, and an inner wall of the soil sample container cooperate to form an accommodation chamber for accommodating a target in-situ frozen soil;
a second thermal conductive disc, located inside the annular support and below the porous plate, wherein a lower end surface of the porous plate, an inner wall of the annular support, and an upper end surface of the second thermal conductive disc cooperate to form a water storage chamber; and
a loading assembly, located above the first thermal conductive disc and configured to apply a load to the target in-situ frozen soil inside the accommodation chamber.

2. The device for measuring a coupling relationship between an ice content and a deformation of a frozen soil according to claim 1, wherein a first flange is provided at a top of the soil sample container; the annular support comprises a second flange and a third flange arranged in sequence from top to bottom; first screws are uniformly distributed in a circumferential direction of the first flange; the first screws pass through the first flange and the second flange in sequence, and each comprise upper and lower ends that are provided with a first nut and a second nut, respectively; second screws are uniformly distributed in a circumferential direction of the second flange; and the second screws pass through the second flange and the third flange in sequence, and each comprise upper and lower ends that are provided with a third nut and a fourth nut, respectively.

3. The device for measuring a coupling relationship between an ice content and a deformation of a frozen soil according to claim 1, wherein the loading assembly comprises a connecting seat, a pressure rod, a weight element, and a pressure plate; the connecting seat is fixed to the soil sample container; the pressure rod comprises one end hinged to the connecting seat and the other end provided with the weight element; an axis of the pressure rod and an axis of the soil sample container intersect and are located in a same vertical plane; the first thermal conductive disc is fixed to the pressure plate; and the pressure plate is slidably connected to the pressure rod.

4. The device for measuring a coupling relationship between an ice content and a deformation of a frozen soil according to claim 3, wherein the loading assembly further comprises a guide plate, a connecting element, a connecting shaft, a guide shaft, and a linear bearing; the guide plate is fixed above the soil sample container; a center of the guide plate coincides with the axis of the soil sample container; the linear bearing is provided at a central part of the guide plate; the pressure rod is axially provided with a sliding groove; two ends of the connecting shaft pass through the sliding groove and are fixed to the connecting element; a bottom of the connecting element is fixed to the connecting shaft; and the other end of the connecting shaft is fixed to the pressure plate.

5. The device for measuring a coupling relationship between an ice content and a deformation of a frozen soil according to claim 3, wherein an end of the pressure rod is axially provided with a mounting groove for limiting the mounting of the weight element.

6. The device for measuring a coupling relationship between an ice content and a deformation of a frozen soil according to claim 1, wherein the first thermal conductive disc and the second thermal conductive discs are structurally identical; the first thermal conductive disc comprises a disc body and a coil; the disc body and the soil sample container are arranged coaxially; the disc body is provided therein with a hollow chamber, and the coil is located inside the hollow chamber; and the coil comprises one end provided with a thermal conductive medium inlet and the other end provided with a thermal conductive medium outlet.

7. The device for measuring a coupling relationship between an ice content and a deformation of a frozen soil according to claim 1, wherein a side wall of the soil sample container is axially provided with mounting holes that are uniformly distributed for mounting frequency domain reflectometry (FDR) sensors.

8. The device for measuring a coupling relationship between an ice content and a deformation of a frozen soil according to claim 1, wherein a base is provided at a bottom of the annular support; and a brake caster is located below the base.

9. The device for measuring a coupling relationship between an ice content and a deformation of a frozen soil according to claim 1, further comprising a distributed sensing optical cable, a pull-out tester, and a fiber optic demodulation device, wherein the distributed sensing optical cable comprises one end fixedly connected to the porous plate and the other end connected to the fiber optic demodulation device; and the pull-out tester and the distributed sensing optical cable are clamped and fixed by a fixture.

10. A method for measuring a coupling relationship between an ice content and a deformation of a frozen soil, based on the device for measuring a coupling relationship between an ice content and a deformation of a frozen soil according to claim 1, and comprising the following steps:
S1: embedding one end of the distributed sensing optical cable in the target in-situ frozen soil, and fixing the one end to the porous plate; and extending the other end of the distributed sensing optical cable outside the soil sample container, and fixing the other end to an external bracket, wherein an axis of the distributed sensing optical cable is parallel to the axis of the soil sample container;
S2: connecting the distributed sensing optical cable to the fiber optic demodulation device; and reading, by the fiber optic demodulation device, temperature information of the distributed sensing optical cable in a length direction, thereby acquiring a temperature distribution and variation of the target in-situ frozen soil in a depth direction;
S3: arranging multiple FDR sensors in an axial direction on the side wall of the soil sample container, and comparing temperature values of the target in-situ frozen soil measured by the multiple FDR sensors with temperature values of the target in-situ frozen soil measured by the distributed sensing optical cable at a same depth;
S4: pulling, by the pull-out tester, the distributed sensing optical cable at a constant rate in different freezing stages of the target in-situ frozen soil, and recording a pulling force and displacement; and monitoring, by the fiber optic demodulation device, an axial strain distribution of the distributed sensing optical cable in the length direction in real time during pulling;
S5: adjusting, by the first thermal conductive disc and the second thermal conductive disc, a temperature of the target in-situ frozen soil;
S6: adjusting, by the loading assembly, a strain of the target in-situ frozen soil in the depth direction; and
S7: measuring a temperature, a water content, and an ice content of the target in-situ frozen soil, as well as a strain distribution and a variation of the target in-situ frozen soil in the depth direction, thereby acquiring a quantitative coupling relationship between the ice content and the deformation.

11. A method for measuring a coupling relationship between an ice content and a deformation of a frozen soil, based on the device for measuring a coupling relationship between an ice content and a deformation of a frozen soil according to claim 2, and comprising the following steps:
S1: embedding one end of the distributed sensing optical cable in the target in-situ frozen soil, and fixing the one end to the porous plate; and extending the other end of the distributed sensing optical cable outside the soil sample container, and fixing the other end to an external bracket, wherein an axis of the distributed sensing optical cable is parallel to the axis of the soil sample container;
S2: connecting the distributed sensing optical cable to the fiber optic demodulation device; and reading, by the fiber optic demodulation device, temperature information of the distributed sensing optical cable in a length direction, thereby acquiring a temperature distribution and variation of the target in-situ frozen soil in a depth direction;
S3: arranging multiple FDR sensors in an axial direction on the side wall of the soil sample container, and comparing temperature values of the target in-situ frozen soil measured by the multiple FDR sensors with temperature values of the target in-situ frozen soil measured by the distributed sensing optical cable at a same depth;
S4: pulling, by the pull-out tester, the distributed sensing optical cable at a constant rate in different freezing stages of the target in-situ frozen soil, and recording a pulling force and displacement; and monitoring, by the fiber optic demodulation device, an axial strain distribution of the distributed sensing optical cable in the length direction in real time during pulling;
S5: adjusting, by the first thermal conductive disc and the second thermal conductive disc, a temperature of the target in-situ frozen soil;
S6: adjusting, by the loading assembly, a strain of the target in-situ frozen soil in the depth direction; and
S7: measuring a temperature, a water content, and an ice content of the target in-situ frozen soil, as well as a strain distribution and a variation of the target in-situ frozen soil in the depth direction, thereby acquiring a quantitative coupling relationship between the ice content and the deformation.

12. A method for measuring a coupling relationship between an ice content and a deformation of a frozen soil, based on the device for measuring a coupling relationship between an ice content and a deformation of a frozen soil according to claim 3, and comprising the following steps:
S1: embedding one end of the distributed sensing optical cable in the target in-situ frozen soil, and fixing the one end to the porous plate; and extending the other end of the distributed sensing optical cable outside the soil sample container, and fixing the other end to an external bracket, wherein an axis of the distributed sensing optical cable is parallel to the axis of the soil sample container;
S2: connecting the distributed sensing optical cable to the fiber optic demodulation device; and reading, by the fiber optic demodulation device, temperature information of the distributed sensing optical cable in a length direction, thereby acquiring a temperature distribution and variation of the target in-situ frozen soil in a depth direction;
S3: arranging multiple FDR sensors in an axial direction on the side wall of the soil sample container, and comparing temperature values of the target in-situ frozen soil measured by the multiple FDR sensors with temperature values of the target in-situ frozen soil measured by the distributed sensing optical cable at a same depth;
S4: pulling, by the pull-out tester, the distributed sensing optical cable at a constant rate in different freezing stages of the target in-situ frozen soil, and recording a pulling force and displacement; and monitoring, by the fiber optic demodulation device, an axial strain distribution of the distributed sensing optical cable in the length direction in real time during pulling; S5: adjusting, by the first thermal conductive disc and the second thermal conductive disc, a temperature of the target in-situ frozen soil;
S6: adjusting, by the loading assembly, a strain of the target in-situ frozen soil in the depth direction; and
S7: measuring a temperature, a water content, and an ice content of the target in-situ frozen soil, as well as a strain distribution and a variation of the target in-situ frozen soil in the depth direction, thereby acquiring a quantitative coupling relationship between the ice content and the deformation.

13. A method for measuring a coupling relationship between an ice content and a deformation of a frozen soil, based on the device for measuring a coupling relationship between an ice content and a deformation of a frozen soil according to claim 4, and comprising the following steps:
S1: embedding one end of the distributed sensing optical cable in the target in-situ frozen soil, and fixing the one end to the porous plate; and extending the other end of the distributed sensing optical cable outside the soil sample container, and fixing the other end to an external bracket, wherein an axis of the distributed sensing optical cable is parallel to the axis of the soil sample container;
S2: connecting the distributed sensing optical cable to the fiber optic demodulation device; and reading, by the fiber optic demodulation device, temperature information of the distributed sensing optical cable in a length direction, thereby acquiring a temperature distribution and variation of the target in-situ frozen soil in a depth direction;
S3: arranging multiple FDR sensors in an axial direction on the side wall of the soil sample container, and comparing temperature values of the target in-situ frozen soil measured by the multiple FDR sensors with temperature values of the target in-situ frozen soil measured by the distributed sensing optical cable at a same depth;
S4: pulling, by the pull-out tester, the distributed sensing optical cable at a constant rate in different freezing stages of the target in-situ frozen soil, and recording a pulling force and displacement; and monitoring, by the fiber optic demodulation device, an axial strain distribution of the distributed sensing optical cable in the length direction in real time during pulling;
S5: adjusting, by the first thermal conductive disc and the second thermal conductive disc, a temperature of the target in-situ frozen soil;
S6: adjusting, by the loading assembly, a strain of the target in-situ frozen soil in the depth direction; and
S7: measuring a temperature, a water content, and an ice content of the target in-situ frozen soil, as well as a strain distribution and a variation of the target in-situ frozen soil in the depth direction, thereby acquiring a quantitative coupling relationship between the ice content and the deformation.

14. A method for measuring a coupling relationship between an ice content and a deformation of a frozen soil, based on the device for measuring a coupling relationship between an ice content and a deformation of a frozen soil according to claim 5, and comprising the following steps:
S1: embedding one end of the distributed sensing optical cable in the target in-situ frozen soil, and fixing the one end to the porous plate; and extending the other end of the distributed sensing optical cable outside the soil sample container, and fixing the other end to an external bracket, wherein an axis of the distributed sensing optical cable is parallel to the axis of the soil sample container;
S2: connecting the distributed sensing optical cable to the fiber optic demodulation device; and reading, by the fiber optic demodulation device, temperature information of the distributed sensing optical cable in a length direction, thereby acquiring a temperature distribution and variation of the target in-situ frozen soil in a depth direction;
S3: arranging multiple FDR sensors in an axial direction on the side wall of the soil sample container, and comparing temperature values of the target in-situ frozen soil measured by the multiple FDR sensors with temperature values of the target in-situ frozen soil measured by the distributed sensing optical cable at a same depth;
S4: pulling, by the pull-out tester, the distributed sensing optical cable at a constant rate in different freezing stages of the target in-situ frozen soil, and recording a pulling force and displacement; and monitoring, by the fiber optic demodulation device, an axial strain distribution of the distributed sensing optical cable in the length direction in real time during pulling;
S5: adjusting, by the first thermal conductive disc and the second thermal conductive disc, a temperature of the target in-situ frozen soil;
S6: adjusting, by the loading assembly, a strain of the target in-situ frozen soil in the depth direction; and
S7: measuring a temperature, a water content, and an ice content of the target in-situ frozen soil, as well as a strain distribution and a variation of the target in-situ frozen soil in the depth direction, thereby acquiring a quantitative coupling relationship between the ice content and the deformation.

15. A method for measuring a coupling relationship between an ice content and a deformation of a frozen soil, based on the device for measuring a coupling relationship between an ice content and a deformation of a frozen soil according to claim 6, and comprising the following steps:
S1: embedding one end of the distributed sensing optical cable in the target in-situ frozen soil, and fixing the one end to the porous plate; and extending the other end of the distributed sensing optical cable outside the soil sample container, and fixing the other end to an external bracket, wherein an axis of the distributed sensing optical cable is parallel to the axis of the soil sample container;
S2: connecting the distributed sensing optical cable to the fiber optic demodulation device; and reading, by the fiber optic demodulation device, temperature information of the distributed sensing optical cable in a length direction, thereby acquiring a temperature distribution and variation of the target in-situ frozen soil in a depth direction;
S3: arranging multiple FDR sensors in an axial direction on the side wall of the soil sample container, and comparing temperature values of the target in-situ frozen soil measured by the multiple FDR sensors with temperature values of the target in-situ frozen soil measured by the distributed sensing optical cable at a same depth;
S4: pulling, by the pull-out tester, the distributed sensing optical cable at a constant rate in different freezing stages of the target in-situ frozen soil, and recording a pulling force and displacement; and monitoring, by the fiber optic demodulation device, an axial strain distribution of the distributed sensing optical cable in the length direction in real time during pulling;
S5: adjusting, by the first thermal conductive disc and the second thermal conductive disc, a temperature of the target in-situ frozen soil;
S6: adjusting, by the loading assembly, a strain of the target in-situ frozen soil in the depth direction; and
S7: measuring a temperature, a water content, and an ice content of the target in-situ frozen soil, as well as a strain distribution and a variation of the target in-situ frozen soil in the depth direction, thereby acquiring a quantitative coupling relationship between the ice content and the deformation.

16. A method for measuring a coupling relationship between an ice content and a deformation of a frozen soil, based on the device for measuring a coupling relationship between an ice content and a deformation of a frozen soil according to claim 7, and comprising the following steps:
S1: embedding one end of the distributed sensing optical cable in the target in-situ frozen soil, and fixing the one end to the porous plate; and extending the other end of the distributed sensing optical cable outside the soil sample container, and fixing the other end to an external bracket, wherein an axis of the distributed sensing optical cable is parallel to the axis of the soil sample container;
S2: connecting the distributed sensing optical cable to the fiber optic demodulation device; and reading, by the fiber optic demodulation device, temperature information of the distributed sensing optical cable in a length direction, thereby acquiring a temperature distribution and variation of the target in-situ frozen soil in a depth direction;
S3: arranging multiple FDR sensors in an axial direction on the side wall of the soil sample container, and comparing temperature values of the target in-situ frozen soil measured by the multiple FDR sensors with temperature values of the target in-situ frozen soil measured by the distributed sensing optical cable at a same depth;
S4: pulling, by the pull-out tester, the distributed sensing optical cable at a constant rate in different freezing stages of the target in-situ frozen soil, and recording a pulling force and displacement; and monitoring, by the fiber optic demodulation device, an axial strain distribution of the distributed sensing optical cable in the length direction in real time during pulling;
S5: adjusting, by the first thermal conductive disc and the second thermal conductive disc, a temperature of the target in-situ frozen soil;
S6: adjusting, by the loading assembly, a strain of the target in-situ frozen soil in the depth direction; and
S7: measuring a temperature, a water content, and an ice content of the target in-situ frozen soil, as well as a strain distribution and a variation of the target in-situ frozen soil in the depth direction, thereby acquiring a quantitative coupling relationship between the ice content and the deformation.

17. A method for measuring a coupling relationship between an ice content and a deformation of a frozen soil, based on the device for measuring a coupling relationship between an ice content and a deformation of a frozen soil according to claim 8, and comprising the following steps:
S1: embedding one end of the distributed sensing optical cable in the target in-situ frozen soil, and fixing the one end to the porous plate; and extending the other end of the distributed sensing optical cable outside the soil sample container, and fixing the other end to an external bracket, wherein an axis of the distributed sensing optical cable is parallel to the axis of the soil sample container;
S2: connecting the distributed sensing optical cable to the fiber optic demodulation device; and reading, by the fiber optic demodulation device, temperature information of the distributed sensing optical cable in a length direction, thereby acquiring a temperature distribution and variation of the target in-situ frozen soil in a depth direction;
S3: arranging multiple FDR sensors in an axial direction on the side wall of the soil sample container, and comparing temperature values of the target in-situ frozen soil measured by the multiple FDR sensors with temperature values of the target in-situ frozen soil measured by the distributed sensing optical cable at a same depth;
S4: pulling, by the pull-out tester, the distributed sensing optical cable at a constant rate in different freezing stages of the target in-situ frozen soil, and recording a pulling force and displacement; and monitoring, by the fiber optic demodulation device, an axial strain distribution of the distributed sensing optical cable in the length direction in real time during pulling;
S5: adjusting, by the first thermal conductive disc and the second thermal conductive disc, a temperature of the target in-situ frozen soil;
S6: adjusting, by the loading assembly, a strain of the target in-situ frozen soil in the depth direction; and
S7: measuring a temperature, a water content, and an ice content of the target in-situ frozen soil, as well as a strain distribution and a variation of the target in-situ frozen soil in the depth direction, thereby acquiring a quantitative coupling relationship between the ice content and the deformation.

18. A method for measuring a coupling relationship between an ice content and a deformation of a frozen soil, based on the device for measuring a coupling relationship between an ice content and a deformation of a frozen soil according to claim 9, and comprising the following steps:
S1: embedding one end of the distributed sensing optical cable in the target in-situ frozen soil, and fixing the one end to the porous plate; and extending the other end of the distributed sensing optical cable outside the soil sample container, and fixing the other end to an external bracket, wherein an axis of the distributed sensing optical cable is parallel to the axis of the soil sample container;
S2: connecting the distributed sensing optical cable to the fiber optic demodulation device; and reading, by the fiber optic demodulation device, temperature information of the distributed sensing optical cable in a length direction, thereby acquiring a temperature distribution and variation of the target in-situ frozen soil in a depth direction;
S3: arranging multiple FDR sensors in an axial direction on the side wall of the soil sample container, and comparing temperature values of the target in-situ frozen soil measured by the multiple FDR sensors with temperature values of the target in-situ frozen soil measured by the distributed sensing optical cable at a same depth;
S4: pulling, by the pull-out tester, the distributed sensing optical cable at a constant rate in different freezing stages of the target in-situ frozen soil, and recording a pulling force and displacement; and monitoring, by the fiber optic demodulation device, an axial strain distribution of the distributed sensing optical cable in the length direction in real time during pulling;
S5: adjusting, by the first thermal conductive disc and the second thermal conductive disc, a temperature of the target in-situ frozen soil;
S6: adjusting, by the loading assembly, a strain of the target in-situ frozen soil in the depth direction; and
S7: measuring a temperature, a water content, and an ice content of the target in-situ frozen soil, as well as a strain distribution and a variation of the target in-situ frozen soil in the depth direction, thereby acquiring a quantitative coupling relationship between the ice content and the deformation.
